(19) United States
(12) Patent Application Publication
(10) Pub. No.: US 2020/0138329 A1
(43) Pub. Date: May 7, 2020

(54) SYSTEMS AND METHODS FOR IMAGING, LOCATING, AND TRACKING A PATIENT

(71) Applicant: Washington University, St. Louis, MO (US)

(72) Inventors: Michael Reilly, St. Louis, MO (US); Nels Knutson, St. Louis, MO (US)

(21) Appl. No.: 16/615,995

(22) PCT Filed: May 22, 2018
(86) PCT No.: PCT/US2018/033859

§ 371 Date: Nov. 22, 2019

Related U.S. Application Data
(60) Provisional application No. 62/509,521, filed on May 22, 2017.

(51) Int. Cl. A61B 5/05; A61N 5/10

(52) U.S. Cl. CPC A61B 5/0507; A61N 2005/1074; A61N 5/1049

(57) ABSTRACT

A setup, imaging, and gating/tracking through holographic topography (S.I.G.H.T.) mapping system includes a mm-wave antenna array and a SIGHT computing device. The mm-wave antenna array is configured to transmit a first plurality of mm-wave signals directed toward a patient positioned on a treatment device, receive a second plurality of mm-wave signal reflected by the patient and the treatment device, and generate at least one output signal based on the second plurality of mm-wave signals. The S.I.G.H.T. computing device includes a processor and a memory coupled to the processor. The S.I.G.H.T. computing device is configured to receive the at least one output signal from the mm-wave antenna array and to process the at least one output signal to generate at least one of: i) a position of a patient relative to the treatment device and ii) a reconstructed image of the patient.

### CROSS-REFERENCE TO RELATED APPLICATION

This International Application claims the benefit of priority to U.S. Provisional Patent Application No. 62/509,521 titled Systems and Methods for Imaging and Locating a Patient for Therapy, filed on May 22, 2017, which is hereby incorporated herein by reference in its entirety.

### FIELD

The field of the disclosure relates generally to imaging, locating, and tracking a patient. More particularly, this disclosure relates to a setup, imaging and gating/tracking through holographic topography (S.I.G.H.T.) mapping system for acquiring real-time imaging of patient receiving therapy from a therapy device without compromising immobilization of the patient.

### BACKGROUND

With at least some known radiation therapy systems, the patient is immobilized relative to part of the therapy system to prevent movement of the patient during treatment. At least some such systems include detection systems to detect the exact location of the patient for effectively providing treatment at desired locations of the patient's body while avoiding application of radiation to undesired locations of the patient's body. The detection systems may also facilitate avoiding collision with components of the systems when the patient or the components of the system are moved. The use of some known systems involves simultaneous large non-coplanar motions of a patient support device and a treatment device, such as a source of radiation, leading to the possibility of collision with the patient. To safely position and provide treatment to the patient, these known systems use various immobilization devices, such as restraining masks, restraining straps, and the like, to immobilize the patient. Some known detection systems, such as visible light image system, infrared image systems, and the like, are unable to image or detect that patient's body through the immobilization devices. Some such systems overcome this difficulty through the use of visible markers positioned on the patient's body or through creating openings in the immobilization devices to allow the detection system to see the patient. Creating openings in the immobilization devices, however, may decrease the effectiveness of the immobilization device, while the use of markers provides a limited number of points only approximating the surface of the patient. Additionally, such known systems often require multiple detectors (e.g., cameras) to be positioned in a number of locations to be capable of consistently detecting the patient during treatment. The inability of capturing the image of the patient increases the possibility of collision of the patient with devices of the systems and reduces the accuracy of providing treatment to specific portions of the patient.

Other systems have been used to image patients to monitor the position of a patient relative to a radiation therapy system. For example, X-ray based systems are able to image the patient through the immobilization devices, but add additional radiation dose to critical organs and are often incompatible with the non-coplanar motions of a patient and radiation gantry motion of some systems.

### BRIEF DESCRIPTION

In one embodiment of the present disclosure, a setup, imaging, and gating/tracking through holographic topography (S.I.G.H.T.) mapping system includes a mm-wave antenna array and a SIGHT computing device. The mm-wave antenna array is configured to transmit a first plurality of mm-wave signals directed toward a patient positioned on a treatment device, receive a second plurality of mm-wave signal reflected by the patient and the treatment device, and generate at least one output signal based on the second plurality of mm-wave signals. The S.I.G.H.T. computing device includes a processor and a memory coupled to the processor. The S.I.G.H.T. computing device is configured to receive the at least one output signal from the mm-wave antenna array and to process the at least one output signal to generate at least one of: i) a position of a patient relative to the treatment device and ii) a reconstructed image of the patient.

In another embodiment of the present disclosure, a method of operating a setup, imaging and gating/tracking through holographic topography (S.I.G.H.T.) mapping system includes transmitting a first plurality of millimeter (mm)-wave signals from an antenna array toward a patient and a treatment device. The method includes receiving a second plurality of mm-wave signals reflected by at least one of the patient and the treatment device at the antenna array. The method includes generating, by the antenna array, at least one output signal based on the second plurality of mm-wave signals. The method includes receiving the at least one output signal at a S.I.G.H.T. computing device. The method includes processing the at least one output signal to generate a position of the patient relative to the treatment device.

In yet another embodiment of the present disclosure, a treatment system includes a platform, a treatment device, and a setup, imaging and gating/tracking through holographic topography (S.I.G.H.T.) mapping system. The platform is configured to support a patient. The treatment device is configured to administer a therapeutic treatment to the patient and to move relative to the platform. The S.I.G.H.T. mapping system includes a millimeter (mm)-wave antenna array configured to transmit a first plurality of mm-wave signals directed toward the patient positioned on the platform. The mm-wave antenna array is configured to receive a second plurality of mm-wave signals reflected by the patient and the platform. The mm-wave antenna array is configured to generate at least one output signal based on the second plurality of mm-wave signals. The S.I.G.H.T. includes a computing device having a processor and a memory coupled to the processor. The S.I.G.H.T. computing device is configured to receive the at least one output signal and process the at least one output signal to generate at least one of i) a position of the patient relative to the treatment device and ii) a reconstructed image of the patient.

### BRIEF DESCRIPTION OF THE DRAWINGS

These and other features, aspects, and advantages of the present disclosure will become better understood when the following detailed description is read with reference to the accompanying drawings in which like characters represent like parts throughout the drawings, wherein:

FIG. 1 is a block diagram of an example computing device;

FIG. 2 is a block diagram of an example setup, imaging and gating/tracking through holographic topography (S.I.G.H.T.) mapping system in accordance with the present disclosure;

FIG. 3 is a lateral-view schematic diagram of the example embodiment of a S.I.G.H.T. mapping system and a treatment system shown in FIG. 2;

FIG. 4 is a schematic block diagram of the S.I.G.H.T. mapping system and the treatment system shown in FIG. 2; and

FIG. 5 is a perspective view of an example treatment system for use with the S.I.G.H.T. mapping system shown in FIGS. 2-4;

FIG. 6 is a perspective view of another example treatment system for use with the S.I.G.H.T. mapping system shown in FIGS. 2-4;

FIG. 7 is a perspective view of an example Gamma Knife treatment system for use with the S.I.G.H.T. mapping system shown in FIGS. 2-4;

FIG. 8 is a perspective view of an example proton therapy treatment system for use with the S.I.G.H.T. mapping system shown in FIGS. 2-4;

FIG. 9 is a front view of another example treatment system for use with the S.I.G.H.T. mapping system shown in FIGS. 2-4; and

FIG. 10 is a front view of the example treatment system shown in FIG. 9.

Unless otherwise indicated, the drawings provided herein are meant to illustrate features of embodiments of the disclosure. These features are believed to be applicable in a wide variety of systems including one or more embodiments of the disclosure. As such, the drawings are not meant to include all conventional features known by those of ordinary skill in the art to be required for the practice of the embodiments disclosed herein.

### DETAILED DESCRIPTION

The example methods and systems described herein provide a setup, imaging and gating/tracking through holographic topography (S.I.G.H.T.) mapping system that is configured to image a surface of a patient through patient immobilization devices with high degree of accuracy while reducing the likelihood of collision among components of the therapy system and the patient. More specifically, the S.I.G.H.T. mapping system described herein includes a millimeter-wave (mm-wave) surface imaging device that provides non-ionizing surface mapping utilizing between about 30 and 300 gigahertz (GHz) mm-waves to image the surface of the patient. The three dimensional mm-wave scan can be performed in a few seconds (in general less than 5 seconds), an amount of time comparable to a diagnostic computerized tomography (CT) scan and significantly shorter than a cone beam computed tomography (CBCT) scan, which usually takes about 60 seconds. The image reconstruction time the S.I.G.H.T. mapping system requires is generally similar to the CT scan, but faster than the CBCT scan.

The reduction of time for scanning and reconstructing the image, as compared to known systems, has an added benefit of significantly reducing the amount of time a given patient spends on or in a treatment device. Additionally, since the S.I.G.H.T. mapping system technology is capable of imaging through clothing (e.g., directly to a person's skin), this technology may be deployed as a real-time motion management strategy. Imaging may take place directly through an immobilization device or mask without any modification to the patient's support devices, thereby preserving its integrity at no-cost to motion monitoring.

Although example embodiments of the S.I.G.H.T. mapping system described herein are used in connection with a therapy system, the S.I.G.H.T. mapping system may also be used with any other suitable system. In some embodiments, the S.I.G.H.T. mapping system is used with another imaging system, or other “modality,” that allows visualization of internal structures, such as a magnetic resonance imaging (MRI) system, an X-ray system, a computed tomography (CT) system, or a cone beam computed tomography (CBCT) system. The S.I.G.H.T. mapping system may be used concurrent with other such modalities, or may be combined with other such modalities during post-processing. During imaging, the S.I.G.H.T. mapping system may be used, for example, to monitor the location and motion of a patient's body surface while the other modalities provide information about internal structures. Images from the S.I.G.H.T. mapping system can be registered, or “fused,” with MRI, X-ray, CT, or CBCT images. Registration of the S.I.G.H.T. data may be achieved using fiducial markers on the patient that appear in both the S.I.G.H.T. image and the other modalities. Such markers are typically adhered or otherwise physically attached to the patient. Alternatively, registration may utilize anatomical markers, or landmarks, that are easily visualized in the S.I.G.H.T. image and the other modalities. In other embodiments, positions and motion of certain internal organs may be inferred from the S.I.G.H.T. data when compared to previously-registered S.I.G.H.T. images or a dynamic time series of images collected using a modality that captures such soft tissue, such as MRI or CT.

Generally, the S.I.G.H.T. mapping system captures the entire patient, while imaging such as MRI, X-ray, CT, or CBCT typically has a narrow field of view. The registered S.I.G.H.T. mapping data provides users of traditional MRI, X-ray, CT, and CBCT systems a more comprehensive picture of the patient that aids in various medical procedures or interventions.

The S.I.G.H.T. mapping system described herein generally includes small transmitting and receiving antennae. For example, in some embodiments, the antennae may be of a diameter less than about eighteen mm. In other embodiments, the antennae may be of any suitable size. In general, the antennae are small relative to the size of a treatment or imaging device. The use of small antennae eliminates the need for the bulky ceiling mounted optical or infrared cameras that are currently used in some imaging systems. Thus, a dynamic use of the antennae and a location flexibility as to where to such antennae may be mounted (e.g., patient treatment couch or directly to the treatment gantry) are enabled. As a result, flexible positioning of non-coplanar patient treatment may be achieved.

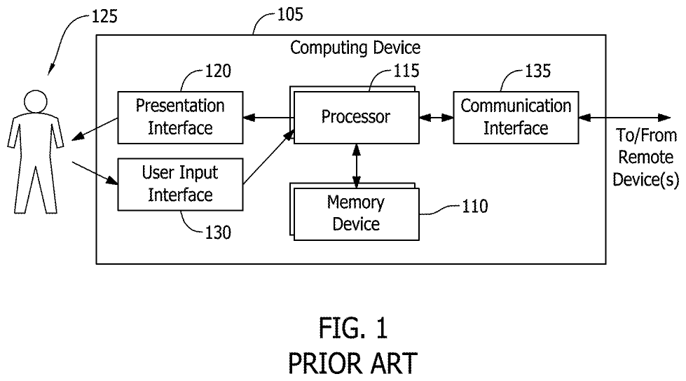

FIG. 1 is a block diagram of an example computing device 105 that may be used to perform monitoring and/or control of a S.I.G.H.T. mapping system (not shown in FIG. 1). Also, in the example embodiment, computing device 105 may monitor and/or control one or more components, systems, processes of a therapy/treatment system associated with the S.I.G.H.T. mapping system, such as gantry rotation drive motors, mm-wave generation devices, antennae, and various monitoring devices (neither shown in FIG. 1). Computing device 105 includes a memory device 110 and a processor 115 operatively coupled to memory device 110 for executing instructions. In some embodiments, executable instructions are stored in memory device 110. Computing device 105 is configurable to perform one or more operations described herein by programming processor 115. For example, processor 115 may be programmed by encoding an operation as one or more executable instructions and providing the executable instructions in memory device 110. In the example embodiment, memory device 110 is one or more devices that enable storage and retrieval of information such as executable instructions and/or other data. Memory device 110 may include one or more computer readable media.

Memory device 110 may be configured to store operational measurements including, without limitation, data acquired by the S.I.G.H.T. mapping system, images generated by the S.I.G.H.T. mapping system, real-time and historical mechanical diagnostic data of the S.I.G.H.T. mapping system, and/or any other type data. Also, memory device 110 includes, without limitation, sufficient data, algorithms, and commands to facilitate monitoring and control of the components within the associated S.I.G.H.T. mapping system.

In some embodiments, computing device 105 includes a presentation interface 120 coupled to processor 115. Presentation interface 120 presents information, such as a user interface and/or an alarm, to a user 125. In some embodiments, presentation interface 120 includes one or more display devices. In some embodiments, presentation interface 120 presents an alarm associated with the associated electric power distribution system being monitored and controlled, such as by using a human machine interface (HMI) (not shown in FIG. 1). Also, in some embodiments, computing device 105 includes a user input interface 130. In the example embodiment, user input interface 130 is coupled to processor 115 and receives input from user 125.

A communication interface 135 is coupled to processor 115 and is configured to be coupled in communication with one or more other devices, such as a sensor or another computing device 105, or any other component of the S.I.G.H.T. mapping system. Communication interface 135 may receive data from and/or transmit data to one or more remote devices. For example, a communication interface 135 of one computing device 105 may transmit an alarm to the communication interface 135 of another computing device 105.

In the example embodiment, control and monitoring of the S.I.G.H.T mapping system is performed with local control devices, such as a localized computing device 105. Alternatively, control and monitoring of the S.I.G.H.T. mapping system may be performed as a portion of a larger, more comprehensive system, such as a portion of a treatment system. Additionally or alternatively, the S.I.G.H.T. mapping system may use one or more computing devices, including computing device 105, that may be located near or at the location of a therapy treatment device or may be remotely located. Additionally or alternatively, he S.I.G.H.T. mapping system may include one or more cloud based components.

In the example embodiment, computing device 105 is but one example of a suitable computing environment and is not intended to suggest any limitation as to the scope of use or functionality of embodiments of the disclosure. Neither should computing device 105 be interpreted as having any dependency or requirement relating to any one or combination of components illustrated.

Embodiments of the present disclosure may be described in the general context of computer code or machine-useable instructions, including computer-executable instructions such as program modules, being executed by a computer or other machine, such as a personal data assistant or other handheld device. Generally, program modules including routines, programs, objects, components, data structures, and the like, refer to code that performs particular tasks or implements particular abstract data types. Embodiments of the disclosure may be practiced in a variety of system configurations, including, but not limited to, hand-held devices, consumer electronics, general purpose computers, specialty computing devices, and the like. Embodiments of the disclosure may also be practiced in distributed computing environments where tasks are performed by remote processing devices that are linked through a communications network.

In a distributed computing environment, program modules may be located in association with both local and remote computer storage media including memory storage devices. The computer useable instructions form an interface to allow a computer to react according to a source of input. The instructions cooperate with other code segments to initiate a variety of tasks in response to data received in conjunction with the source of the received data.

Computing device 105 typically includes a variety of computer-readable media. Computer-readable media can be any available media that can be accessed by computing device 105 and includes both volatile and non-volatile media, removable and non-removable media. By way of example, and not limitation, computer-readable media may comprise computer storage media and communication media. Computer storage media includes both volatile and non-volatile, removable and non-removable media implemented in any method or technology for storage of information such as computer-readable instructions, data structures information, program modules or other data program modules or other data. Computer storage media includes, but is not limited to, RAM, ROM, EEPROM, flash memory or other memory technology, CD-ROM, digital versatile disks (DVD) or other optical disk storage, magnetic cassettes, magnetic tape, magnetic disk storage or other mag-netic storage devices, or any other medium which can be used to store the desired information and which can be accessed by computing device 105. Computer storage media does not include signals per se. Communication media typically embodies computer-readable instructions, data structures, program modules, or other data in a modulated data signal such as a carrier wave or other transport mechanism and includes any information delivery media. The term “modulated data signal” means a signal that has one or more of its characteristics set or changed in such a manner as to encode information in the signal. By way of example, and not limitation, communication media includes wired media such as a wired network or direct-wired connection, and wireless media such as acoustic, RF, infrared and other wireless media. Combinations of any of the above should also be included within the scope of computer-readable media.

User input interface 130 allow computing device 105 to be logically coupled to other devices, some of which may be built in. User input interface 130 may include, for example, without limitation, a keyboard, a pointing device, a mouse, a stylus, a touch sensitive panel, such as, without limitation, a touch pad or a touch screen, and/or an audio input interface, such as, without limitation, a microphone. A single component, such as a touch screen, may function as both a display device of presentation interface 120 and user input interface 130.

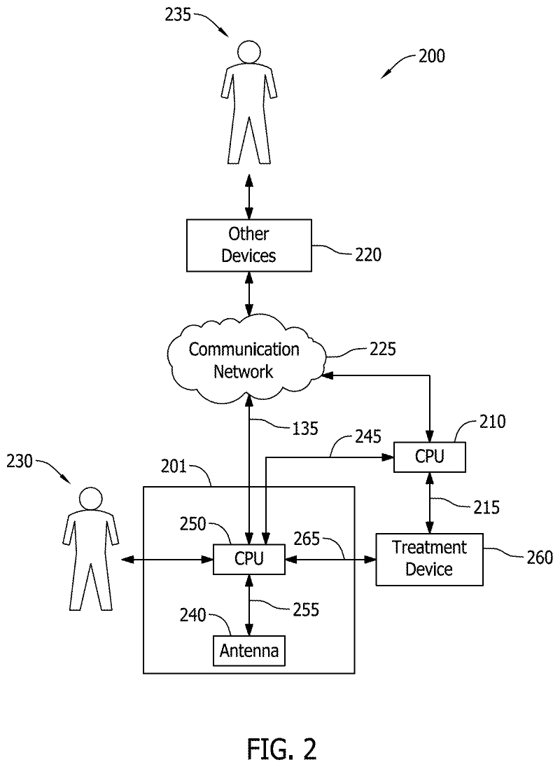

FIG. 2 is a block diagram of a portion of a treatment system 200 (sometimes referred to herein as a “therapy system”) including a S.I.G.H.T. mapping system 201. Treatment system 200 includes a central processing unit (CPU) 210, other devices 220, communication network 225, at least one antenna 240, CPU 250, and treatment device 260 (also referred to sometimes as a “therapy device”). Treatment system 200 may be operated by at least operator 230 and/or operator 235. In some embodiments, CPU 210 is configured to execute monitoring and control algorithms and monitoring and control logic. CPU 210 is configured to control treatment device 260, and receive from and transmit data to S.I.G.H.T. mapping system 201. CPU 210 may be coupled to other devices 220 via a communication network 225, where, in some embodiments, communication network 225 includes the Internet. The other devices 220 may include, for example, a remote computing device, a remote monitor, a remote memory device, etc. CPU 210 is coupled to CPU 250. CPU 250 is configured to provide patient location data to CPU 210 for use in controlling treatment device 260. In certain embodiments, CPU 210 and CPU 250 may be each a computing device 105. In other CPU 210 and 250 may be a single computing device 105 and the actions described as performed by CPU 210 and CPU 250 are performed by the single computing device 105. In yet other embodiments, CPU 210 and CPU 250 are any other suitable controllers for operating as described herein.

CPU 250 interacts with a first operator 230, e.g., without limitation, via user input interface 130 and/or presentation interface 120 (both shown in FIG. 1). In one embodiment, CPU 250 presents patient location information from S.I.G.H.T. system 201 and information derived from the patient location information to operator 230. Other devices 220 interact with a second operator 235, e.g., without limitation, via user input interface 130 and/or presentation interface 120. In some embodiments, other devices 220 present patient location information, alarms, and/or other operational information to second operator 235. As used herein, the term “operator” includes any person in any capacity associated with operating and maintaining treatment system 200 and S.I.G.H.T. system 201, including, without limitation, imaging technicians, maintenance technicians, doctors, and nurses.

In some embodiments, other devices 220 include one or more storage devices that are any computer-operated hardware suitable for storing and/or retrieving data, for example, and without limitation, multiple storage units such as hard disks or solid state disks in a redundant array of inexpensive disks (RAID) configuration, a storage area network (SAN), and/or a network attached storage (NAS) system.

S.I.G.H.T. system 201 includes at least one antenna 240 coupled to CPU 250 through at least one input/output (I/O) channel 255. The at least one antenna 240 transmits and receives mm-wave signals to detect physical characteristics of a patient being treated by treatment device 260. In some embodiments, each antenna 240 is operable to transmit and receive mm-wave signals. In other embodiments, the at least one antenna 240 is at least a pair of antennae 240. One antenna 240 of such a pair is configured to transmit mm-wave signals and the other antenna 240 is configured to receive mm-wave signals. The at least one antenna 240 repeatedly, e.g., periodically, continuously, and/or upon request, transmits operational measurement readings at the time of measurement to CPU 250. CPU 250 receives and processes the operational measurement readings. In one embodiment, such data may be transmitted across network 225 and may be accessed by any authorized device capable of accessing network 225 including, without limitation, desktop computers, laptop computers, and personal digital assistants (PDAs) (neither shown). In another embodiment, such data may be transmitted to CPU 210 through at least one I/O channel 245.

S.I.G.H.T. mapping system 201 is adaptable to any suitable oncology treatment modalities. S.I.G.H.T. mapping system 201 is coupled to treatment device 260, which may represent different treatment modalities. Treatment device 260 may include treatment devices for Gamma Knife radio-surgery, proton therapy, linear acceleration (Linac) therapy, CT scan, cyberknife, tomography, or the like. Treatment device 260 is in communication with CPU 210 through I/O channel 265. Treatment device 260 may also be in communication with communication network 225. Treatment device 260 may further be in communication with CPU 250 through I/O channel 265. In certain embodiments, S.I.G.H.T. mapping system 201 may be part of treatment device 260. For example, antenna 240 may be attached to at least portion of treatment device 260. In other embodiments, S.I.G.H.T. mapping system 201 is implemented as a stand-alone system. For example, antenna 240 may be placed in a location having access to treatment device 260, but antenna 240 is not attached to treatment device 260.

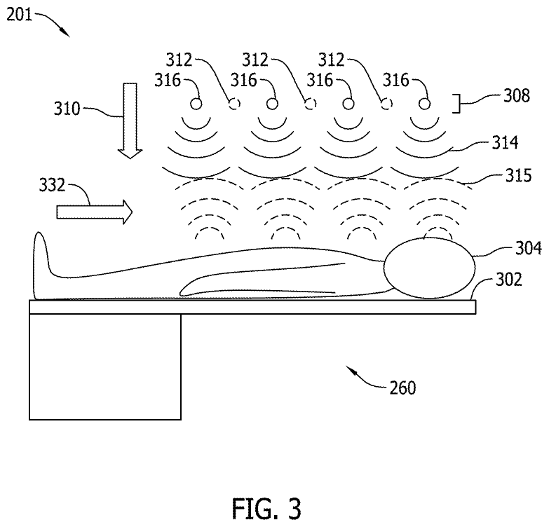

FIG. 3 is a lateral view of an embodiment of S.I.G.H.T. mapping system 201. Referring to FIG. 3, treatment device 260 includes a platform 302 (e.g., patient treatment couch) for positioning a patient 304 in treatment device 260.

Antenna array 308 (e.g., mm-wave array) transmits mm-waves 314 toward patient 304 positioned on platform 302. In some embodiments, antenna array 308 includes a dynamic array implemented by either physically moving antennae 240 relative to patient 304 to produce a three dimensional image or by using a fixed array with digital imaging of individual and sequential antenna to acquire an equivalent volumetric image. In the example embodiment, antenna array 308 includes a plurality of antennae 240 that may be compact and lightweight (e.g., each antenna may have a diameter of less than about eighteen mm). In the example embodiment, antennae 240 include two types of antennae, transmitter antennae 312, and receiver antennae 316. Transmitter antennae 312 transmit mm-wave signals 314 toward patient 304 and receiver antennae 316 receives mm-wave signals 315 reflected back from patient 304 and any other non-absorbing item struck by the transmitted mm-wave signals 314. Although seven antennae 240 are shown in FIG. 3, antenna array 308 may include more or fewer antennae 240. Although antenna array 308 must include at least one transmitter antenna 312 and one receiver antenna 316, in some embodiments, a single antenna 240 may perform as both transmitter antenna 312 and receiver antenna 316.

In certain embodiments, antenna array 308 is installed at a fixed position within a treatment room. In other embodiments, antenna array 308 is installed on a mechanical rotating array that attaches to treatment device 260 and rotates around at least part of patient 304. In other embodiments, antenna array 308 is installed on a gantry (not shown) of treatment device 260. During a scan that acquires mm-wave signals, a gantry and antenna array 308 rotate within a plane in the direction of arrow 310 and around a longitudinal axis, or z-axis, of patient 304 about a center of rotation, while patient 304 is moved through gantry in along the z-axis 332 perpendicular to the plane of rotation. In yet other embodiments, during a scan, antenna array 308 is at a fixed position within treatment device while the gantry rotates as described above.

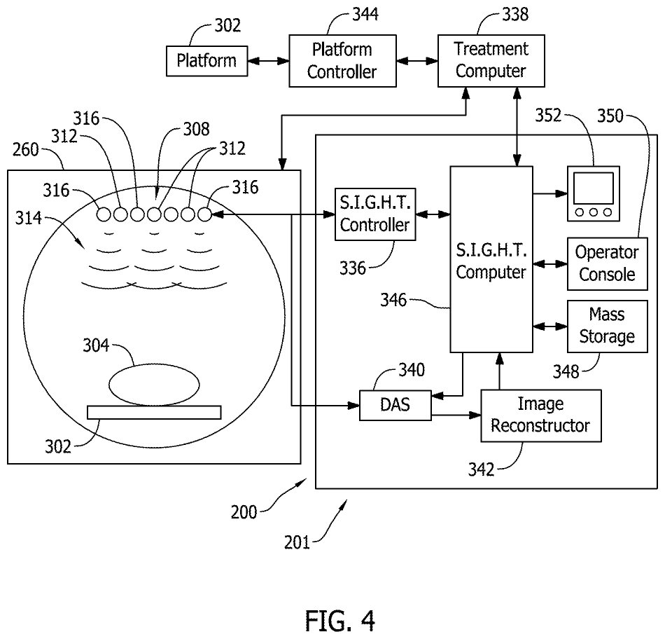

FIG. 4 is a schematic diagram of treatment system 200 (shown in FIG. 2) that includes S.I.G.H.T. mapping system 201 (shown in FIG. 2 and FIG. 3). Antenna array 308 is controlled by S.I.G.H.T. mapping system 201, which may include a S.I.G.H.T. controller 336, a S.I.G.H.T computer 346, a data acquisition system (DAS) 340, an image reconstructor 342, a mass storage system 348, an operator console 350, and a display device 352. In some embodiments, S.I.G.H.T computer 356 is computer 105 (shown in FIGS. 1 and 2) and/or CPU 250 (shown in FIG. 2). Also, in some embodiments, mass storage system 348 is one of other devices 220 (shown in FIG. 2). Further, in some embodiments, operator console 350 is user input interface 130 (shown in FIG. 1). Moreover, in some embodiments, display device 352 is presentation interface 120 (shown in FIG. 1). Communication between the various system elements of FIG. 4 is depicted by arrowhead lines that illustrate a means for either signal communication or mechanical operation, depending on the system element involved.

S.I.G.H.T. controller 336 controls antenna array 308. More specifically, S.I.G.H.T. controller 336 provides power and timing signals to antenna array 308, which transmits mm-wave signals 314 to patient 304 using transmitter antennas 312 and receives mm-wave signals 314 reflected by patient 304 or platform 302 using receiver antennae 316. Receiver antennae 316 generate one or more output signals representing the received mm-wave signals. Data acquisition system 340 acquires the output signals from antenna array 308 and converts the signals to a digital format for subsequent processing. In certain embodiments, S.I.G.H.T. computer 346 determines a position of the patient relative to, for example, treatment device 260. In certain embodiments, image reconstructor 342 receives the digital signals from data acquisition system 340 and performs an image reconstruction process that involves filtering the digital signal data using a reconstruction algorithm.

S.I.G.H.T. computer 346 is in communication with S.I.G.H.T. controller 336 whereby control signals are sent from S.I.G.H.T. computer 346 to controllers 336 and information is received from controller 336 by S.I.G.H.T. computer 346. S.I.G.H.T. computer 346 also provides commands and operational parameters to data acquisition system 340 and receives reconstructed image data from image reconstructor 342. The reconstructed image data is stored by S.I.G.H.T. computer 346 in mass storage system 348 for subsequent retrieval. A physician or other operator may create a treatment plan based on a reconstructed image, or reconstructed image data. In certain embodiments, one or more subsequently reconstructed images, based on subsequently acquired data from antenna array 308, may be compared to the prior reconstructed image for the purpose of adjusting the previously determined treatment plan.

An operator (either first operator 230 or second operator 235, both shown in FIG. 2) interfaces with S.I.G.H.T. computer 346 through operator console 350, which may include, for example, a keyboard and a graphical pointing device, and receives output, such as, for example, a reconstructed image, control settings and other information, on display device 352. S.I.G.H.T. computer 346 is also in communication with a treatment computer 338. In some embodiments, treatment computer 338 is computer 105 (shown in FIGS. 1 and 2) and/or CPU 210 (shown in FIG. 2).

S.I.G.H.T. computer 346 is in further communication with treatment computer 338 to enable receiving and transmission of data with treatment computer 338. In some embodiments, treatment computer 338 is in communication with a platform controller 344 whereby control signals are sent from treatment computer 338 to platform controller 344 and information is received from platform controller 344 by treatment computer 338. Platform controller 344 controls the position and direction of platform 302. In certain embodiments, when S.I.G.H.T. computer 346 determines a distance between patient 304 and treatment device 260 falls below a threshold distance, S.I.G.H.T. computer 346 communicates with treatment computer 338 to instruct platform controller 344 to stop movement of platform 302 and patient 304 relative to treatment device 260. In certain embodiments, S.I.G.H.T. computer 346 may instruct treatment computer 338 to modify or stop the treatment itself based on the distance between patient 304 and treatment device 260, or based on relative motion between patient 304 and treatment device 260.

Communication among the various system elements may be achieved through a hardwired or a wireless arrangement. S.I.G.H.T. computer 346 may be a standalone computer or a network computer and may include instructions in a variety of computer languages for use on a variety of computer platforms and under a variety of operating systems. Other examples of S.I.G.H.T. computer 346 include a system having one or more microprocessors, microcontrollers, or other equivalent processing devices capable of executing commands of computer readable data or program for executing a control algorithm. In order to perform the prescribed functions and desired processing, as well as the associated computations, e.g., the execution of Fourier analysis algorithm(s) and the control processes prescribed herein, computer 346 includes, without limitation, a processor(s) 115 and memory 110 (both shown in FIG. 1), storage, register(s), timing, interrupt(s), communication interfaces, and input/output signal interfaces, as well as combinations including at least one of the foregoing. For example, computer 346 may include input signal filtering to enable accurate sampling and conversion or acquisitions of such signals from communications interfaces. As described herein, example embodiments are implemented through computer-implemented processes and apparatuses for practicing those processes.

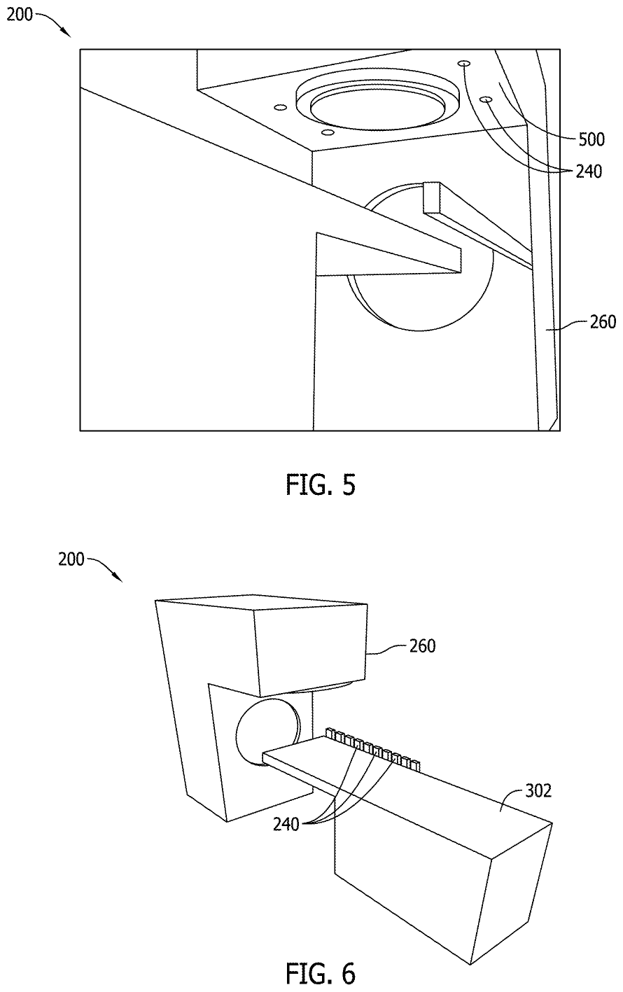

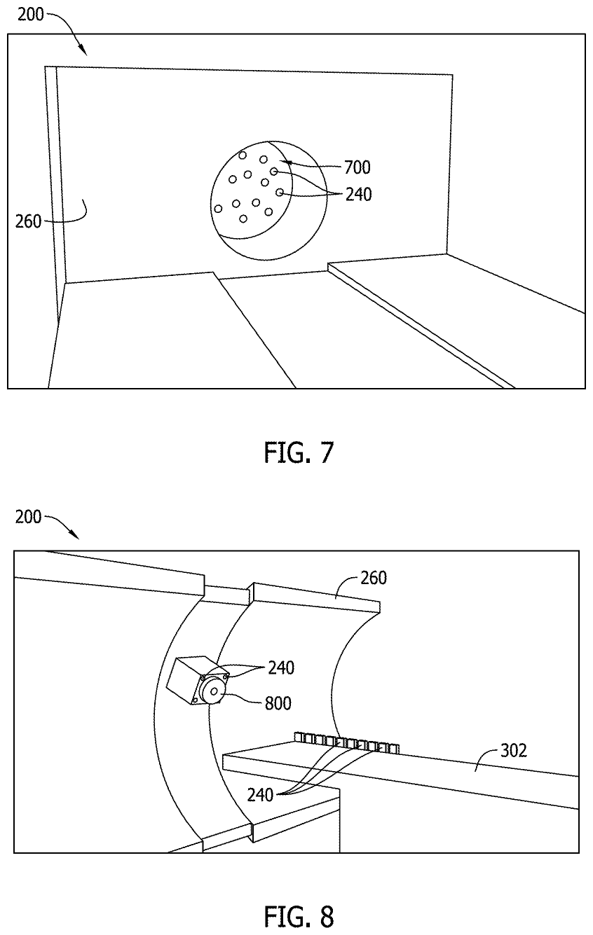

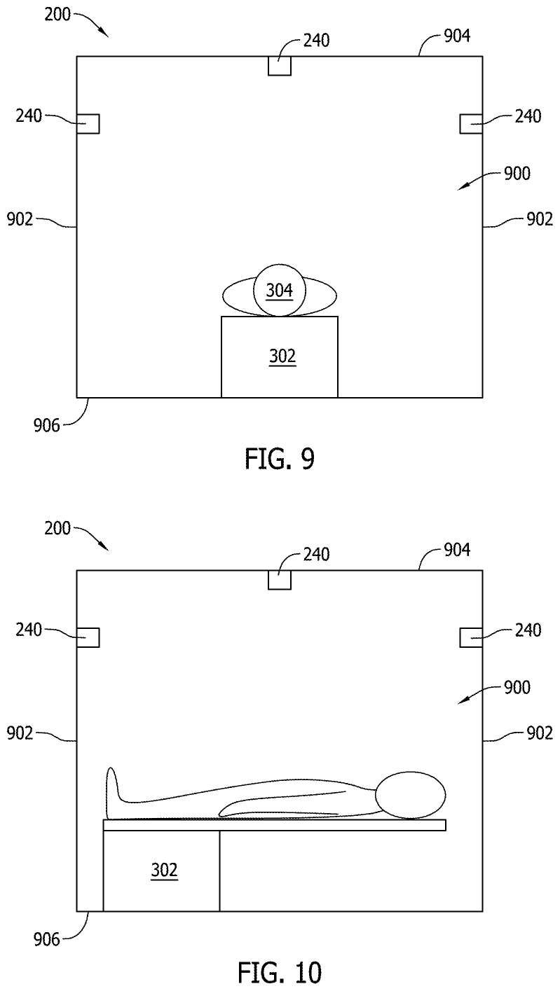

FIGS. 5-10 are perspective views of various treatment systems 200 including S.I.G.H.T. mapping system 201. Referring to FIG. 5, treatment device 260 is a Linac treatment device. Antennae 240 are installed on a gantry 500 of treatment device 260. In FIG. 6, treatment device 260 is a Linac treatment device with antennae 240 installed adjacent the platform 302. FIG. 7 is an embodiment of system 200 in which treatment device 260 is a Gamma Knife treatment device where antennae 240 are installed within dome 700. In FIG. 8, treatment device 260 is a proton therapy device where antennae 240 are on platform 302. In this embodiment, antennae 240 are also mounted on the exit nozzle 800, also referred to as a window, of the proton therapy device. The embodiments shown in FIGS. 5-8 all include antennae 240 mounted to treatment device 260. In other embodiments, antennae 240 may be attached external to the treatment device. FIG. 9 and FIG. 10 are front and side views of an example treatment system 200 with antennae 240 mounted around a treatment room 900. Antennae 240 may be mounted to walls 902, ceilings 904, floors 906, or any other fixed structure in treatment room 900. Antennae 240 are configured to transmit one or more signals to a treatment device's platform 302 where patient 304 is located. Antennae 240 are also configured to receive one or more signals from platform 302 where patient 304 is located.

A technical effect of the systems and methods described herein includes at least one of: (a) increasing the speed of volumetric scanning using existing diagnostic scan systems; (b) eliminating additional imaging dose; (c) reducing real-time distance to the surface of a patient without compromising support devices; (d) facilitating unparalleled access to the patient within the radiation treatment vault; (e) improving flexibility of the system design that enables a dynamic array of implementations by either mechanically scanning a patient where the array physically moves or by constructing a fixed array where digitally scanning of individual and sequential antenna acquire the equivalent volumetric image; (f) increasing accuracy enabling the reduction of dose to critical organs and escalating volume dose to tumor without compromising patient safety; (g) localizing and monitoring patients in any treatment position while preventing potential collisions; (h) increasing scan speeds while reducing patient treatment time; (i) providing clinicians with continuous current volumetric image tracking tools to improve clinical efficacy; (j) using mm-wave imaging for patient setup and immobilization in radiation therapy, the motion management of patients in radiation therapy, and detection of in-room distance measurements in radiation therapy, and (k) using mm-wave imaging for monitoring of active gantry position for collision detection in radiation therapy, monitoring patients in SRS treatment using Gamma Knife, monitoring patients in proton therapy treatment rooms, performing guided biopsy treatment, mapping of oral cavities in dental implant design, conducting in patient CT simulation, and using mm-wave antennae as external fiducial tracking markers in radiation therapy.

Example embodiments of the S.I.G.H.T. mapping systems, and methods of operating such systems and devices are not limited to the specific embodiments described herein, but rather, components of systems and/or steps of the methods may be utilized independently and separately from other components and/or steps described herein. For example, the methods may also be used in combination with other systems that use rapidly rotating components requiring accurate scanning, and are not limited to practice with only the S.I.G.H.T. mapping systems and methods as described herein.

Although specific features of various embodiments of the disclosure may be shown in some drawings and not in others, this is for convenience only. In accordance with the principles of the disclosure, any feature of a drawing may be referenced and/or claimed in combination with any feature of any other drawing.

Some embodiments involve the use of one or more electronic or computing devices. Such devices typically include a processor, processing device, or controller, such as a general purpose central processing unit (CPU), a graphics processing unit (GPU), a microcontroller, a reduced instruction set computer (RISC) processor, an application specific integrated circuit (ASIC), a programmable logic circuit (PLC), a field programmable gate array (FPGA), a digital signal processing (DSP) device, and/or any other circuit or processing device capable of executing the functions described herein. The methods described herein may be encoded as executable instructions embodied in a computer readable medium, including, without limitation, a storage device, and/or a memory device. Such instructions, when executed by a processing device, cause the processing device to perform at least a portion of the methods described herein. The above examples are example only, and thus are not intended to limit in any way the definition and/or meaning of the term processor and processing device.

This written description uses examples to disclose the embodiments, including the best mode, and also to enable any person skilled in the art to practice the embodiments, including making and using any devices or systems and performing any incorporated methods. The patentable scope of the disclosure is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal language of the claims.

What is claimed is:

1. A setup, imaging and gating/tracking through holographic topography (S.I.G.H.T.) mapping system comprising:
a millimeter (mm)-wave antenna array configured to:
transmit a first plurality of mm-wave signals directed toward a patient positioned on a treatment device;
receive a second plurality of mm-wave signals reflected by the patient and the treatment device; and
generate at least one output signal based on the second plurality of mm-wave signals; and
a S.I.G.H.T. computing device comprising a processor and a memory coupled to the processor, the S.I.G.H.T. computing device configured to:
receive the at least one output signal; and
process the at least one output signal to generate at least one of i) a position of a patient relative to the treatment device and ii) a reconstructed image of the patient.

2. The S.I.G.H.T. mapping system of claim 1, wherein the mm-wave antenna array includes a first plurality of transmit antennas and a second plurality of receive antennas.

3. The S.I.G.H.T. mapping system of claim 1, wherein the S.I.G.H.T. computing device is further configured to process the at least one output signal to determine a distance between the position of the patient and a position of at least one component of the treatment device.

4. The S.I.G.H.T. mapping system of claim 3, wherein the S.I.G.H.T. computing device is coupled to the treatment device and is further configured to instruct the treatment device to stop movement of at least one component of the treatment device when the distance between the position of the patient and the position of the at least one component of the treatment device is less than a threshold distance.

5. The S.I.G.H.T. mapping system of claim 1, wherein the mm-wave antenna array is mounted on the treatment device.

6. The S.I.G.H.T. mapping system of claim 5, wherein the mm-wave antenna array is mounted on a gantry of the treatment device.

7. The S.I.G.H.T. mapping system of claim 5, wherein the mm-wave antenna array is integrated into at least one component of the treatment device.

8. The S.I.G.H.T. mapping system of claim 1, wherein the S.I.G.H.T. computing device is further configured to determine an amount of movement of at least one portion of the patient relative to the treatment device based on the at least one output signal.

9. The S.I.G.H.T. mapping system of claim 8, wherein the S.I.G.H.T. computing device is coupled to the treatment device and is further configured to instruct the treatment device to stop providing treatment when the determined amount of movement of the at least one portion of the patient relative to the treatment device exceeds a threshold amount.

10. The S.I.G.H.T. mapping system of claim 1, wherein the S.I.G.H.T. computing device is further configured to compare the reconstructed image of the patient to a previously acquired image of the patient.

11. The S.I.G.H.T. mapping system of claim 8, wherein the S.I.G.H.T. computing device is coupled to the treatment device and is further configured to instruct the treatment device, based on the comparison of the reconstructed image of the patient to the previously acquired image of the patient, to adjust a treatment plan that was based on the previously acquired image of the patient.

12. The S.I.G.H.T. mapping system of claim 1, wherein the S.I.G.H.T. computing device is coupled to the treatment device and is further configured to control operation of the treatment device based on a previously determined treatment plan and the reconstructed image of the patient.

13. A method of operating a setup, imaging and gating/tracking through holographic topography (S.I.G.H.T.) mapping system, the method comprising:
transmitting a first plurality of millimeter (mm)-wave signals from an antenna array toward a patient and a treatment device;
receiving a second plurality of mm-wave signals reflected by at least one of the patient and the treatment device at the antenna array;
generating, by the antenna array, at least one output signal based on the second plurality of mm-wave signals;
receiving the at least one output signal at a S.I.G.H.T. computing device; and
processing the at least one output signal to generate a position of the patient relative to the treatment device.

14. The method of claim 13 further comprising processing the at least one output signal to generate a reconstructed image of the patient.

15. A treatment system, comprising:
a platform configured to support a patient;
a treatment device configured to:
administer a therapeutic treatment to the patient; and
move relative to the platform; and
a setup, imaging and gating/tracking through holographic topography (S.I.G.H.T.) mapping system comprising:
a millimeter (mm)-wave antenna array configured to:
transmit a first plurality of mm-wave signals directed toward the patient positioned on the platform;
receive a second plurality of mm-wave signals reflected by the patient and the platform; and
generate at least one output signal based on the second plurality of mm-wave signals; and
a S.I.G.H.T. computing device comprising a processor and a memory coupled to the processor, the S.I.G.H.T. computing device configured to:
receive the at least one output signal; and
process the at least one output signal to generate at least one of i) a position of the patient relative to the treatment device and ii) a reconstructed image of the patient.
